(19) United States
(12) Patent Application Publication
(10) Pub. No.: US 2019/0000309 A1
(43) Pub. Date: Jan. 3, 2019

(54) LARYNGEAL CAMERA UNIT, AND LARYNGEAL CAMERA SET

(71) Applicant: SCALAR CORPORATION, Tokyo (JP)

(72) Inventor: Masao Yamamoto, Tokyo (JP)

(21) Appl. No.: 16/066,376

(22) PCT Filed: Dec. 28, 2016
(86) PCT No.: PCT/JP2016/089147

§ 371 Date: Jun. 27, 2018

(30) Foreign Application Priority Data
Dec. 28, 2015 (JP) 2015-257485

(51) Int. Cl. A61B 1/267; A61B 1/05; A61B 1/055; A61M 1/00; A61B 1/00; A61B 1/06

(52) U.S. Cl. CPC A61B 1/267; A61B 1/05; A61B 1/055; A61B 1/0676; A61B 1/00142; A61B 1/0669; A61M 1/008

(57) ABSTRACT

It is an object to provide an inexpensive technology capable of alleviating pain of a patient during phlegm suction. A laryngeal camera unit (200) is used in combination with a portable device including a lens, which forms a part of a camera, and a light. The laryngeal camera unit (200) includes an illumination light transmission member (223) and an image light transmission member (222), both of which are rod-shaped and are to be inserted in an oral cavity. Illumination light from the light of the portable device is transmitted to a larynx by the illumination light transmission member (223), and image light from the larynx is transmitted from behind a uvula to the portable device by the image light transmission member (222). A user performs the phlegm suction while checking an image displayed on a display of the portable device.

### TECHNICAL FIELD

The present invention relates to a laryngeal camera configured to image a larynx, or relates to a laryngeal camera unit which is to be combined with an existing camera to form the laryngeal camera. The present invention relates to, for example, a laryngeal camera or a laryngeal camera unit, which may be used during phlegm suction.

### BACKGROUND ART

Particularly for elderly people, phlegm suction is inevitable. Therefore, phlegm suction devices are used in medical institutions and nursing homes, for example, and are increasingly popular among ordinary households.

Simply put, a phlegm suction device is like a compact vacuum cleaner.

In broad terms, the phlegm suction device includes a tube made of a flexible material, a suction bottle, which is a container connected to a proximal end of the tube, and a suction pump, which is an air pump configured to create negative pressure in the tube through the suction bottle.

When the suction device is used, a distal end of the tube (to be more precise, the tube is generally formed of a tubular tube on the proximal end side of the tube, and a tubular catheter that is connected to a distal end of the tube and is thinner than the tube, but with the tube and the catheter both being tubular and with no advantage in distinguishing the two in relation to the invention of this application, the tube and the catheter are collectively referred to herein as the “tube”) is inserted in an oral cavity or a nasal cavity of a patient such that the distal end of the tube reaches the place where phlegm collects (which is generally the region of a larynx, for example, near vocal cords). Under this state, the negative pressure is created in the tube through the suction bottle by the suction pump to guide the phlegm from the distal end to the proximal end of the tube and collect the phlegm in the suction bottle.

### SUMMARY

### Technical Problem

Suction devices are essential and are widespread because there is no problem with their function of sucking phlegm per se. However, there are still problems to be solved.

When a suction device is used as described above, a tube is inserted in an oral cavity or a nasal cavity. This may cause pain for a patient. When the tube is inserted from the oral cavity, the patient may experience an emetic response. When the tube is inserted from the nasal cavity, the tube often rubs against a sensitive mucous membrane in the nasal cavity to cause stronger pain for the patient than in the case where the tube is inserted from the oral cavity. In addition, when the tube is inserted from the nasal cavity, it is relatively easy to guide a distal end of the tube to a phlegm suction position of the patient by guiding the tube with the use of an upward curve of an upper surface of the nasal cavity in a plane along a midline of a face (that said, the distal end of the tube may sometimes be caught by fine irregularities on the inside of the nasal cavity, and it is actually not that easy for the distal end of the tube to reach the phlegm suction position), but when the tube is inserted from the oral cavity, with the oral cavity having no effective curve for guiding the tube downward, and with a tongue sticking out upward from below (in this application, the concepts of up and down in a body of the patient are different from those in an actual situation of usage of the phlegm suction device, but correspond to up and down directions under a state in which the patient stands upright) in the oral cavity of the patient interfering, it is difficult to guide the distal end of the tube to the phlegm suction position of the patient. Therefore, when the phlegm suction device is used, there is often no choice but to insert the tube from the nasal cavity, which causes stronger pain for the patient.

Another factor that increases the pain for the patient is as follows.

The suction device is often used not by the patient himself or herself but by medical personnel, for example, or by a caregiver for the patient in households, but whoever uses the suction device invariably does not know the position in the body of the patient at which the phlegm is present. Therefore, the user of the suction device moves the tube inserted in the oral cavity or in the nasal cavity back and forth or rotates the tube around its axis again and again until the distal end of the tube reaches the place where the phlegm is present. Then, every time the user thinks that the distal end of the tube has reached the place where the phlegm is present, the user drives a suction pump. This operation is repeated again and again until the phlegm is sucked.

Such operation of the tube at a guess, so to speak, increases the pain for the patient.

In order to eliminate such operation of the tube at a guess, it is only required that an operator of the suction device may visually identify a position of the distal end of the tube. To that end, in medical institutions, phlegm suction is also performed while visually identifying the position of the distal end of the tube with the use of an endoscope of a type to be inserted in the nasal cavity (the phlegm suction position seen with such an endoscope is near a larynx, and hence such an endoscope is essentially a laryngeal camera), for example.

However, an endoscope is expensive. Therefore, it is almost impossible for phlegm suction devices to spread such that endoscopes may be used during phlegm suction for all of a large number of patients in medical institutions. For the same reason, it is difficult for endoscopes to spread to households.

Observation near the larynx is required for checking a tumor in the region of the larynx or checking motor function near the larynx, and hence there is a demand for laryngeal cameras regardless of the use for the phlegm suction. However, there remains a problem in proliferation of the laryngeal cameras mainly for the reason of cost.

It is a problem to be solved by the invention of this application to provide a technology capable of proliferating laryngeal camera inexpensively.

### Solution to Problem

In order to solve the above-mentioned problem, the inventor of this application proposes the following invention.

According to one embodiment of the invention of this application, there is provided a laryngeal camera unit, which is to be combined with a portable device to form a laryngeal camera, the portable device being configured to be held by hand, and including an image sensor and a lens, which are configured to take an image of image light from a subject, and a light, which is configured to emit illumination light for illuminating the subject, the laryngeal camera being configured to take an image of a larynx.

The laryngeal camera unit includes: rod-shaped image light transmission means for receiving, from a distal end of the image light transmission means, the image light from the larynx, transmitting the image light along a long direction of the image light transmission means, and irradiating the lens of the portable device with the image light from a proximal end of the image light transmission means to allow the image sensor to take the image, the distal end being configured to reach behind a uvula when the larynx is imaged; rod-shaped illumination light transmission means for receiving, from a proximal end of the illumination light transmission means, the illumination light from the light of the portable device, transmitting the illumination light along a long direction of the illumination light transmission means, and irradiating the larynx with the illumination light from a distal end of the illumination light transmission means, the distal end being configured to reach behind the uvula when the larynx is imaged, the illumination light transmission means being configured to extend along the image light transmission means at least in a predetermined range on the distal end side of the illumination light transmission means; and a main body portion, to which the proximal end of the image light transmission means and the proximal end of the illumination light transmission means are attached, which includes fixing means for detachably fixing the main body portion to the portable device under a state of being positioned in a predetermined relative positional relationship, and which is to be fixed to the portable device so that the proximal end of the image light transmission means is positioned in front of and in proximity to the lens of the portable device, and so that the proximal end of the illumination light transmission means is positioned in front of and in proximity to the light of the portable device.

Such a laryngeal camera unit is to be combined with a portable device to form a laryngeal camera. The portable device is a smartphone or a tablet computer, for example. Such a portable device is already widespread, and hence when the portable device is to be combined with the laryngeal camera unit according to one embodiment of the invention of this application to form the laryngeal camera, a user is required to purchase only the laryngeal camera unit. The laryngeal camera unit, which may form the laryngeal camera when combined with the portable device, may be manufactured and sold with not so much cost. Meanwhile, when a user of a phlegm suction device or other such user does not have a portable device, the user may purchase a laryngeal camera set obtained by combining the laryngeal camera unit and a portable device, and the portable device in this case has an appearance resembling a smartphone or a tablet computer, for example, and is only required to include an image sensor, a light, and preferably a display. Such a portable device that does not require functions such as communication, electronic mail, and internet browsing may be manufactured and sold with not so much cost as compared to a smartphone or a tablet computer, and may be manufactured and sold very inexpensively at least as compared to an endoscope, which is exclusive goods. Therefore, even when it is required to manufacture and sell not the laryngeal camera unit but such a laryngeal camera set as described above, a small economic burden is placed on the user of the laryngeal camera.

In addition, such a laryngeal camera unit may be basically formed only of the following three minimal components: image light transmission means for transmitting image light from a larynx to a lens of the portable device; illumination light transmission means for transmitting illumination light from the light of the portable device to the larynx; and a main body portion, to which the image light transmission means and the illumination light transmission means are attached, which includes fixing means for detachably fixing the main body portion to the portable device, and which is configured to uniquely determine, when the main body portion is fixed to the portable device, a relative positional relationship between a proximal end of the image light transmission means and the lens of the portable device, and a relative positional relationship between a proximal end of the illumination light transmission means and the light of the portable device. Therefore, the image sensor and the light are not required in the laryngeal camera unit according to one embodiment of the invention of this application per se, and hence it is not required to mount electric and electronic parts in the laryngeal camera unit according to one embodiment of the invention of this application. This also contributes significantly to the reduction in cost for manufacturing and selling the laryngeal camera unit according to one embodiment of the invention of this application.

Further, the portable device as described above normally also includes the display. Therefore, the user of the phlegm suction device or other such user of the laryngeal camera may observe the larynx in real time, for example, while checking an image of the larynx taken by the image sensor and displayed on the display substantially in real time.

The laryngeal camera may be a phlegm suction camera, which is to be used to assist in phlegm suction by imaging a phlegm suction position. In this case, when the portable device includes the display, the user of the phlegm suction device may operate the tube while watching the image of the phlegm suction position. If a suction pump of the phlegm suction device is operated when the distal end of the tube reaches the phlegm, the phlegm suction may be performed reliably in an extremely short time, and hence pain experienced by a patient may be minimized. When the portable device does not include any display, the image taken by the image sensor may be displayed on a display of another display device external to the portable device as a matter of course.

When a distal end of an image light transmission member of the laryngeal camera is moved, the laryngeal camera may image a part other than the larynx, for example, a pharynx or an esophagus. The term “laryngeal camera” as used in the invention of this application means a camera capable of imaging at least a part of the larynx when a distal end of an image light transmission member of the camera is appropriately moved, and a camera capable of imaging another part by moving a distal end of an image light transmission member of the camera is also included in the “laryngeal camera” in one embodiment of the invention of this application.

Both of the image light transmission means and the illumination light transmission means are rod-shaped as described above. The term “rod-shaped” as used herein only means that a length in a long direction is longer than horizontal and vertical lengths of a cross section perpendicular to the long direction, and includes a case of being as thin as generally expressed as “linear”.

The image light transmission means and the illumination light transmission means, both of which are rod-shaped, may be bent to correspond to a curve of a palate in a vertical direction of the palate. The fact that the image light transmission means and the illumination light transmission means are bent means that, when the image light transmission means and the illumination light transmission means are inserted in the oral cavity, it is easy for the image light transmission means and the illumination light transmission means to reach the back of the oral cavity of the patient, and more specifically, for the distal end of each of the image light transmission means and the illumination light transmission means to reach behind the uvula more easily while avoiding a tongue of the patient. Therefore, the laryngeal camera including the laryngeal camera unit is less likely to cause the pain due to emetic response for the patient when in use.

In the laryngeal camera unit according to one embodiment of the invention of this application, the image light transmission means and the illumination light transmission means may be fixed to each other. When relative positions of the image light transmission means and the illumination light transmission means are fixed, the image light transmission means and the illumination light transmission means may be prevented from being separated more than necessary from each other, and hence a bundle of the image light transmission means and the illumination light transmission means may be made thinner. This, as a matter of course, contributes to alleviation of the pain experienced by the patient due to the emetic response when the laryngeal camera including the laryngeal camera unit is used.

Moreover, it is required that the larynx, which is a site from which the image light is emitted and taken in from a distal end of the image light transmission means, be illuminated with the illumination light radiated from a distal end of the illumination light transmission means. To that end, it is preferred that a relative positional relationship between the distal end of the image light transmission means and the distal end of the illumination light transmission means be fixed. When the image light transmission means and the illumination light transmission means are fixed to each other, the relative positional relationship between the distal end of the image light transmission means and the distal end of the illumination light transmission means is advantageously fixed.

The image light transmission means and the illumination light transmission means may be housed in a case, which is configured to house the image light transmission means and the illumination light transmission means under a state of allowing light to pass through the distal end and the proximal end of each of the image light transmission means and the illumination light transmission means. When the case is used, it becomes easy to use a cover, which is to be described later. Moreover, the image light transmission means and the illumination light transmission means may be housed in the case to fix the image light transmission means and the illumination light transmission means to each other.

The case may be bent to correspond to a curve of a palate in a vertical direction of the palate. This configuration provides the effect similar to that obtained when the image light transmission means and the illumination light transmission means are bent, that is, the effect that the laryngeal camera including the laryngeal camera unit is less likely to cause the pain due to the emetic response for the patient when in use.

In the laryngeal camera unit according to one embodiment of the invention of this application, at least one of the image light transmission means or the illumination light transmission means may include a tube fixing member configured to detachably fix a flexible tube, which is included in a phlegm suction device, under a state in which a distal end of the flexible tube is positioned in an imaging range of the image sensor. The laryngeal camera formed with the use of the laryngeal camera unit in this case is the phlegm suction camera, and is used to assist in the phlegm suction. Without limiting, the phlegm suction position is often the larynx or the region of the larynx, and hence it is required for the laryngeal camera to be able to image at least a part of at least the larynx.

The laryngeal camera formed with the use of the laryngeal camera unit for use in such application is, simply put, configured to image the distal end of the tube and allow checking of a relative positional relationship of the distal end of the tube with respect to the phlegm. In other words, it is preferred that the distal end of the tube be always captured in the image taken by the image sensor, and that a relative positional relationship between the distal end of the tube and the distal end of the image light transmission means be fixed. This is possible when the laryngeal camera unit includes such a tube fixing member as described above, and the operation in which the distal end of the tube reaches the phlegm or the movement of the distal end of the tube may be conveniently performed at the same time with the operation or the movement of the laryngeal camera including the laryngeal camera unit according to one embodiment of the invention of this application.

At least one of the image light transmission means or the illumination light transmission means may include a tube fixing member configured to detachably fix a flexible tube, which is included in a phlegm suction device, and the tube fixing member may be configured to fix the flexible tube with the flexible tube in a portion fixed by the tube fixing member being bent at a curvature that is larger than curvatures imparted to the image light transmission means and the illumination light transmission means so that a distal end of the flexible tube is headed to the larynx. As described above, the oral cavity has no structure that is appropriately bent to guide the distal end of the tube downward in the body of the patient, that is, in a direction toward the larynx or the region of the larynx, which is the phlegm suction position. When the tube is bent at a curvature that is larger than curvatures imparted to the image light transmission means and the illumination light transmission means at the portion of the tube fixing member, the distal end of the tube may be guided downward in the body of the patient, that is, toward the phlegm suction position, by the tube fixing member.

When the laryngeal camera unit includes the case, the tube fixing member may be provided to the case. That is, the case may include a tube fixing member configured to detachably fix a flexible tube, which is included in a phlegm suction device, under a state in which a distal end of the flexible tube is positioned in an imaging range of the image sensor. The effect obtained with this configuration is similar to that obtained in the case where the tube fixing member is provided to at least one of the image light transmission means or the illumination light transmission means.

When the case includes a tube fixing member configured to detachably fix a flexible tube, which is included in a phlegm suction device, the tube fixing member may be configured to fix the flexible tube with the flexible tube in a portion fixed by the tube fixing member being bent at a curvature that is larger than a curvature imparted to the case so that a distal end of the flexible tube is headed to the larynx. The effect obtained with this configuration is similar to that obtained in the case where the tube fixing member, which is provided to at least one of the illumination light transmission means or the image light transmission means, is configured to fix the tube with the tube in the portion fixed by the tube fixing member being bent at a curvature that is larger than curvatures imparted to the image light transmission means and the illumination light transmission means so that the distal end of the tube is headed to the larynx.

The laryngeal camera unit may further include a cover, which is configured to cover at least a portion of the case to be inserted in an oral cavity, which is detachably attachable to the case, and in which at least portions for allowing passage of the image light and the illumination light are transparent. The case is inserted in the oral cavity as described above. When the laryngeal camera unit includes the cover configured to cover the laryngeal camera unit, the cover may be disposal in particular such that the laryngeal camera unit is always kept clean.

It is sufficient when the cover in this case is configured to cover at least the portion of the case to be inserted in the oral cavity, and it is not required for the cover to cover the entire case.

The cover may include a tube fixing member configured to detachably fix a flexible tube, which is included in a phlegm suction device, under a state in which a distal end of the flexible tube is positioned in an imaging range of the image sensor. The effect obtained in this case is similar to that obtained in the case where the tube fixing member is provided to the case. As with the case in which the tube fixing member is provided to the case, the tube fixing member in this case may be configured, as a matter of course, to fix the tube with the tube in the portion fixed by the tube fixing member being bent at a curvature that is larger than a curvature imparted to the case so that the distal end of the tube is headed to the larynx.

### BRIEF DESCRIPTION OF THE DRAWINGS

FIG. 1(A) is a perspective view as seen from a back side and FIG. 1(B) is a perspective view as seen from a front side of a portable device for use in combination with a laryngeal camera unit according to a first embodiment of the present invention;

FIG. 2(A) is a perspective view as seen from a back side and FIG. 2(B) is a perspective view as seen from a front side of the laryngeal camera unit according to the first embodiment;

FIG. 3 is an enlarged perspective view of a case included in the laryngeal camera unit illustrated in FIG. 2;

FIG. 4(A) is an enlarged view of a distal end of an image light transmission member included in the laryngeal camera unit illustrated in FIG. 2, FIG. 4(B) is an enlarged view of a distal end of an illumination light transmission member included in the laryngeal camera unit, and FIG. 4(C) is an enlarged view of another example of the distal end of the illumination light transmission member included in the laryngeal camera unit;

FIG. 5 is a cross-sectional view for illustrating in enlargement a part of head and neck of a human body;

FIG. 6 is a view for illustrating a state of use of a laryngeal camera formed by combining the laryngeal camera unit illustrated in FIG. 2 and the portable device;

FIG. 7 is a perspective view of a cover for use in combination with a laryngeal camera unit according to a second embodiment of the present invention;

FIG. 8 is a view for illustrating a state of use of a laryngeal camera formed by combining the laryngeal camera unit according to the second embodiment and a portable device; and

FIG. 9(A) is a perspective view as seen from a back side and FIG. 9(B) is a perspective view as seen from a front side of a laryngeal camera unit according to a third embodiment of the present invention.

### DESCRIPTION OF EMBODIMENTS

Now, first to third embodiments of the present invention are described with reference to the drawings.

In the description of the embodiments, common objects are denoted by common reference symbols, and common description is omitted in some cases.

### First Embodiment

First, a portable device, which is to be combined with a laryngeal camera unit according to the first embodiment to form a laryngeal camera, is described. The laryngeal camera formed by combining the laryngeal camera unit according to the first embodiment with the portable device is, without limiting, a phlegm suction camera to be used for the purpose of assisting in phlegm suction. This is also true for the second embodiment. It should be noted, however, that the laryngeal camera obtained by combining the laryngeal camera unit according to the first embodiment and the portable device may be used simply to observe a larynx and its vicinities without being combined with a phlegm suction device.

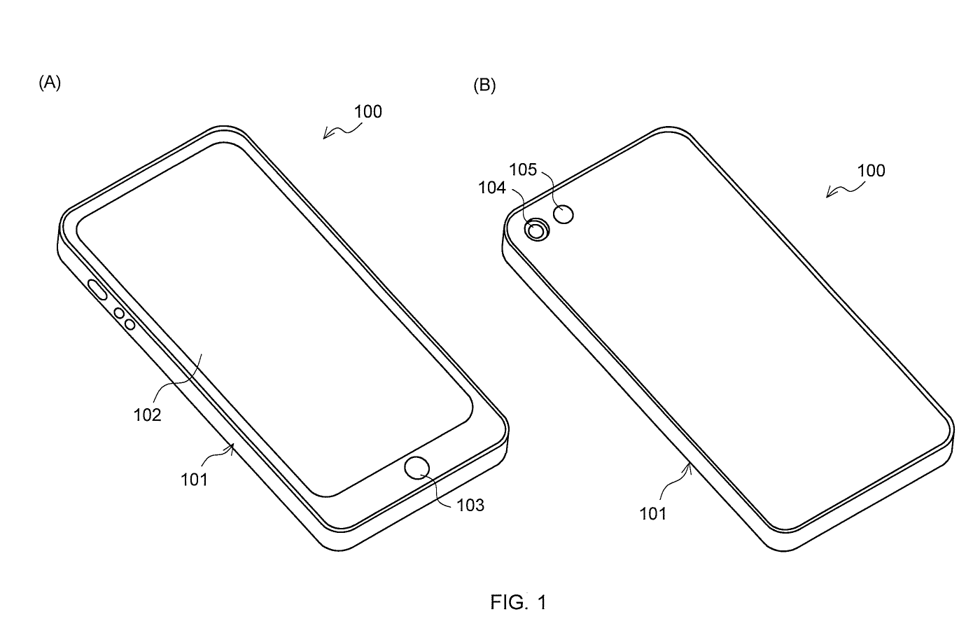

A portable device 100 is illustrated in FIG. 1. FIG. 1(A) is a perspective view as seen from the back and FIG. 1(B) is a perspective view as seen from the front of the portable device.

The portable device 100 may be a publicly-known or well-known portable device, and is described simply. The portable device 100 is compact enough to be portable, and it is an essential condition for the portable device 100 to include a camera, which is capable of taking a moving image, and a light, both of which are to be described later. It is required for the portable device 100 to be compact so that the laryngeal camera including the portable device 100 is easy to operate, and is easy to operate preferably with one hand. It is required for the portable device 100 to include the camera capable of taking a moving image and the light so that the camera and the light are used as a camera and a light of the laryngeal camera.

Moreover, it is preferred that the portable device 100 include a display. This is because the moving image taken by the camera is displayed on the display so that the display can be used as a monitor of the laryngeal camera. It should be noted, however, that when the portable device 100 does not include the display, a display of a display device external to the portable device 100 may be used as the monitor of the laryngeal camera instead of the display of the portable device 100. In that case, it is required to send data of the moving image from the portable device 100 to the display device, but the portable device 100 and the display device may be suitably connected to each other through a USB cable, for example.

Without limiting, the portable device 100 is a smartphone or a tablet computer, and is a smartphone in the first embodiment. The smartphone may be a smartphone that is commercially available. Examples of the smartphone include iPhone (trademark) series manufactured and sold by Apple Japan, Inc. Examples of the tablet computer include iPad (trademark) series and iPod (trademark) series manufactured and sold by Apple Japan, Inc.

The portable device 100 includes a housing 101 formed in a thin and substantially rectangular parallelepiped shape. The housing 101 is compact enough to be held by one hand.

The portable device 100 includes a display 102 on a back surface of the housing 101. The display 102 is configured to display an image, and may be a publicly-known or well-known display, for example, a liquid crystal display.

The portable device 100 also includes an input device 103 on the back surface of the housing 101. The input device 103 is used to operate the portable device 100. The input device 103 may be physical keys, but when the display 102 is a touch panel, the display 102 may also have a part of the function of the input device 103.

On a front side of the housing 101 of the portable device 100, a lens 104 is exposed. The lens 104 forms a part of the camera of the portable device 100. As is well known, an image sensor (not shown) is located behind the lens 104. The image sensor is a charge-coupled device (CCD) or a complementary metal oxide semiconductor image sensor (CMOS), for example, and is configured to be able to take a moving image. The image taken by the image sensor is displayed on the display 102 substantially in real time by a publicly-known or well-known mechanism.

The portable device 100 also includes a light 105 on a front surface of the housing 101. It is only required that the light 105 emit illumination light. An example of the light 105 is an LED.

Next, the laryngeal camera unit is described.

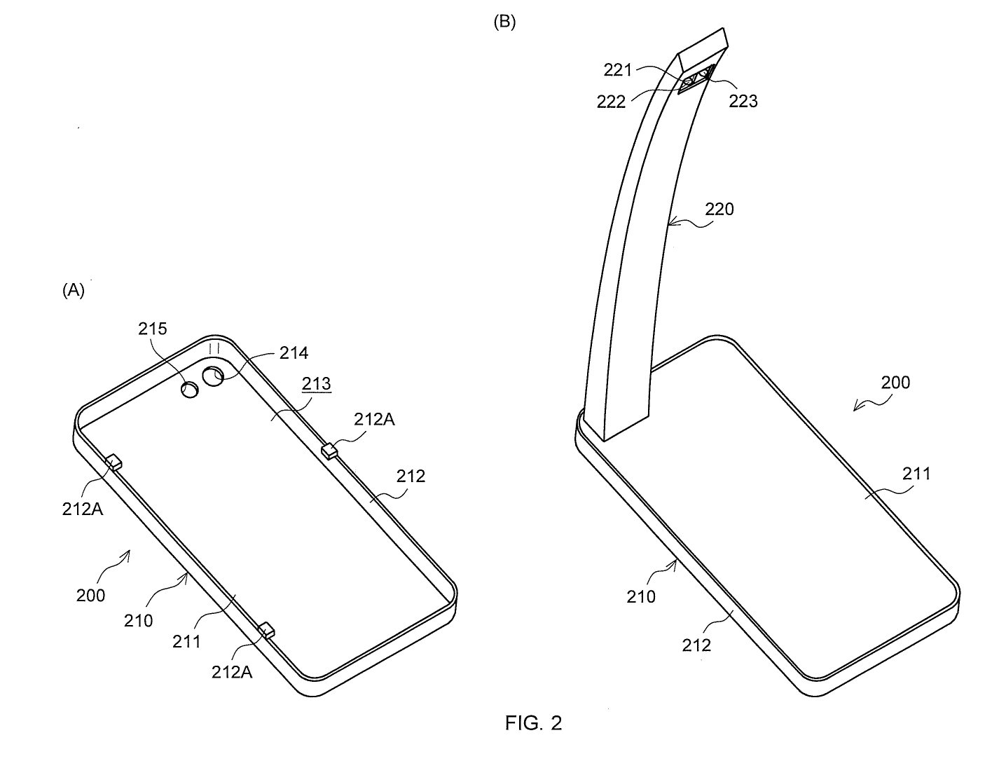

In FIG. 2, a laryngeal camera unit 200 is illustrated. FIG. 2(A) is a perspective view of the laryngeal camera unit 200 as seen from the back, and FIG. 2(B) is a perspective view of the laryngeal camera unit 200 as seen from the front.

The laryngeal camera unit 200 includes a main body portion 210. The main body portion 210 is configured to detachably fix the laryngeal camera unit 200 to the portable device 100.

Without limiting, the main body portion 210 in the first embodiment is made of resin. Moreover, without limiting, the main body portion 210 in the first embodiment includes a plate portion 211, which is a substantially rectangular plate having a large area, and a side plate portion 212, which is a plate that surrounds the periphery of the plate portion 211, sticks out on a back surface side of the plate portion 211, and has the same height from the plate portion 211 in all parts of the side plate portion 211. The main body portion 210 has a recess 213 as a space surrounded by the plate portion 211 and the side plate portion 212 on a back side of the main body portion 210.

The recess 213 has a size and shape that correspond to the housing 101 of the portable device 100, and is configured to house the portable device 100 entirely in a stable state. When combined with the laryngeal camera unit 200 to form the laryngeal camera, the portable device 100 is housed in the recess 213 in an orientation in which the back surface of the portable device 100 is exposed from the recess 213.

Around the recess 213 of the side plate portion 212, locking claws 212A, which are small plate-like members that stick out toward a center direction of the recess 213, are formed. When the portable device 100 is housed in the recess 213, the portable device 100 is locked by the locking claws 212A under a state of not falling off the recess 213, to thereby achieve removable fixing of the portable device 100 to the laryngeal camera unit 200. It should be noted, however, that it is not always required that the removable fixing between the portable device 100 and the laryngeal camera unit 200 be achieved by forming the recess 213 in the main body portion 210 of the laryngeal camera unit 200. For example, the removable fixing between the portable device 100 and the laryngeal camera unit 200 may be achieved by forming, in the main body portion 210, a groove or a hole having such size and shape that allow the portable device 100 to be inserted in the main body portion 210 by translating the portable device 100 in a long direction of the portable device 100, and that prevent the portable device 100 inserted in the main body portion 210 from dropping off (for example, a groove having a cross-sectional substantially C shape, and having both ends slightly sticking out in parallel with respect to the bottom of the groove), and sliding and inserting the portable device 100 into the groove or the hole in the main body portion 210 along the long direction of the portable device 100, for example. Needless to say, the removable fixing between the portable device 100 and the laryngeal camera unit 200 may be achieved by another publicly-known or well-known method.

In a portion of the plate portion 211 that corresponds to the lens 104 of the portable device 100 when the portable device 100 is housed in the recess 213, a first aperture 214 is formed. The first aperture 214 is, without limiting, circular. In order to form a passage for image light to be described later, the first aperture 214 is formed not to cover the lens 104 of the portable device 100, which is housed in the recess 213, with the plate portion 211, and may have a diameter that is larger than that of the lens 104 or is not required to be circular as long as the lens 104 is not covered with the plate portion 211.

In a portion of the plate portion 211 that corresponds to the light 105 of the portable device 100 when the portable device 100 is housed in the recess 213, a second aperture 215 is formed. The second aperture 215 is, without limiting, circular. In order to form a passage for illumination light to be described later, the second aperture 215 is formed not to cover the light 105 of the portable device 100, which is housed in the recess 213, with the plate portion 211, and may have a diameter that is larger than that of the light 105 or is not required to be circular as long as the light 105 is not covered with the plate portion 211. Further, the first aperture 214 and the second aperture 215 may be integrated.

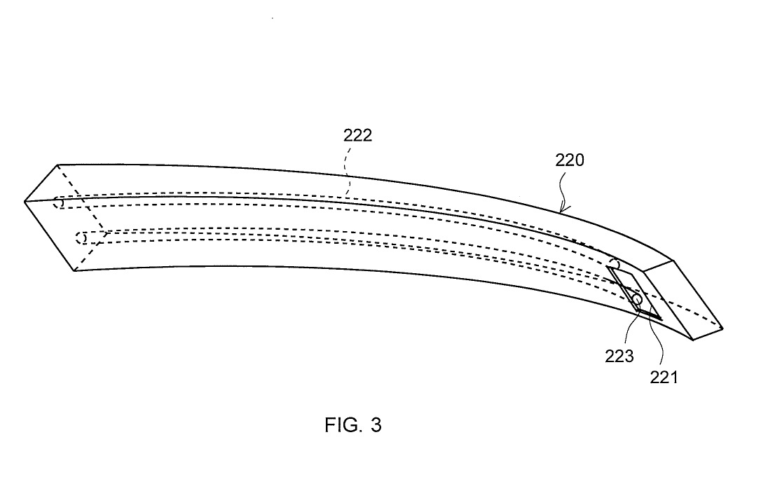

To the main body portion 210, a case 220 is attached. The case 220 is illustrated in enlargement in FIG. 3.

The case 220 is made of resin as with the main body portion 210, and is formed integrally with the main body portion 210, for example. The case 220 has a slender and substantially parallele piped shape, and has its width unchanged from a proximal end to a distal end thereof, but has its thickness gradually thinned toward the distal end. Moreover, the case 220 is bent downward in the use of the laryngeal camera toward the distal end. The proximal end side of the case 220 is opened.

The case 220 has such a length that, when the case 220 is inserted in an oral cavity during the use of the laryngeal camera, the distal end of the case 220, and more specifically, a window formed in the case 220, which is to be described later, can reach behind the uvula.

On a lower side near the distal end of the case 220, a window 221 is formed. The window 221 is a passage for the image light and the illumination light for allowing the image light and the illumination light, which are to be described later, to pass through the case 220 without being affected by the case 220. The window 221 is formed by boring a part of the case 220, without limiting, in a rectangular shape, and fitting and fixing a transparent plate having a uniform thickness to the opening. It is preferred that the part in which the transparent plate and the opening are brought into contact with each other be watertight so as to prevent saliva and the like of a patient from entering the case 220 during the use of the laryngeal camera. It should be noted, however, that the window 221 may be divided into the following two: a window for the image light and a window for the illumination light. Moreover, when the entire case 220 is formed of a transparent resin, it is not required to form the window 221 in the case 220. In that case, however, in order to ensure that the case 220 does not function as a lens, it is preferred that at least a part of the case 220 through which the image light passes has a uniform thickness.

Inside the case 220, an image light transmission member 222 and an illumination light transmission member 223 are arranged. Both of the image light transmission member 222 and the illumination light transmission member 223 are long rod-like members, and have substantially the same length as the length from the proximal end to the distal end of the case 220.

The image light transmission member 222 is configured to transmit the image light that has entered the image light transmission member 222 from a distal end to a proximal end thereof. Such transmission of the image light is required to be transmission of the image light from the larynx under a state in which an image can be taken by the image sensor included in the portable device 100. The image light transmission member 222 is a graded index (GI) type rod lens, for example. The image light transmission member 222 is rod-shaped and bent along the case 220. The image light transmission member 222 is fixed to the inside of the case 220 with the use of, for example, a rib (not shown) formed inside the case 220. The periphery of the proximal end of the image light transmission member 222 is fixed to the periphery of the first aperture 214 formed in the plate portion 211 of the main body portion 210 so as not to affect the transmission of the image light. When the portable device 100 is fixed to the recess 213 of the main body portion 210, a surface at the proximal end of the image light transmission member 222 is positioned right in front of the lens 104 such that an optical axis of the image light transmission member 222 matches an optical axis of the lens 104 of the portable device 100. Moreover, the distal end of the image light transmission member 222 faces the window 221 of the case 220. In other words, when the case 220 is inserted in the oral cavity for use of the laryngeal camera, the distal end of the image light transmission member 222 reaches behind the uvula. The image light transmission member 222 may be formed of a publicly-known image guide, which is formed by bundling a large number of optical fibers, instead of the rod lens. When the large number of optical fibers has the same relative positional relationship at both ends of the image guide, the image light can be transmitted from a distal end to a proximal end of the image guide under a state in which an image can be taken correctly by the image sensor of the portable device 100. When such an image guide is adopted, it becomes easy to bend the image light transmission member 222.

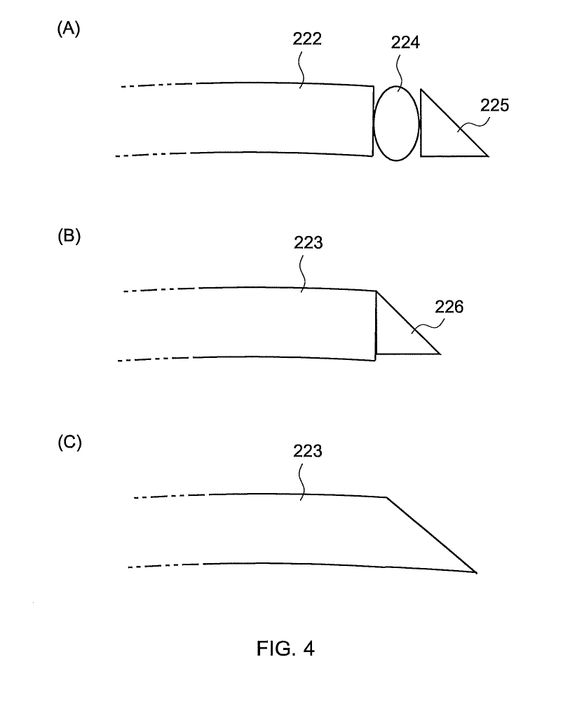

Without limiting, in the first embodiment, on the distal end side of the image light transmission member 222, as illustrated in FIG. 4(A), an objective lens 224 is provided. The objective lens 224 has the function of increasing an angle of view (to 60° or more, preferably about 70°, for example) of the image light transmission member 222, which is the rod lens, which is difficult to increase. As a result, the image sensor can image a wide range including at least a part of the larynx with the image light transmission member 222. Moreover, on the further distal end side of the objective lens 224 of the image light transmission member 222 in the first embodiment, a reflecting member 225 is provided. The reflecting member 225 is configured to reflect the image light to change an angle of the image light, to thereby allow the image light from a lower side of the inside of the body of the patient to enter the distal end of the image light transmission member 222. The reflecting member 225 is a mirror or a prism, for example. When the distal end of the image light transmission member 222 can be directed to a direction near a glottis, which is the larynx, the reflecting member 225 is not required, but when it is difficult to direct the distal end to the direction near the glottis, the reflecting member 225 is used to allow the image light from the larynx to enter a surface at the distal end of the image light transmission member 222 through the reflecting member 225.

On the proximal end side of the image light transmission member 222, an optical element for allowing the image light to appropriately enter the lens 104 of the portable device 100 may be provided. Such an optical element may converge the image light in the range of a pupil of the lens 104 at a solid angle that is the same as or larger than an angle of view of the lens 104, for example. The optical element in this case is one or more lenses, for example.

The illumination light transmission member 223 is configured to transmit the illumination light that has entered the illumination light transmission member 223 from a proximal end to a distal end thereof, and irradiate the larynx with the illumination light. The illumination light transmission member 223 may be one optical fiber or a bundle of a plurality of optical fibers, for example. A GI type rod lens may be used as the illumination light transmission member 223. Moreover, a mirror-finished metal tube may be used as the illumination light transmission member 223, for example, such that an inner peripheral surface of the metal tube reflects light. The illumination light transmission member 223 is rod-shaped and bent along the case 220. The illumination light transmission member 223 is fixed to the inside of the case 220 with the use of, for example, a rib (not shown) formed inside the case 220. In other words, the illumination light transmission member 223 is fixed to the image light transmission member 222 via the case 220. The periphery of the proximal end of the illumination light transmission member 223, for example, is fixed to the periphery of the second aperture 215 formed in the plate portion 211 of the main body portion 210 so as not to affect the transmission of the illumination light. When the portable device 100 is fixed to the recess 213 of the main body portion 210, a surface at the proximal end of the illumination light transmission member 223 is positioned right in front of the light 105 of the portable device 100. As a result, the illumination light from the light 105 is allowed to enter the illumination light transmission member 223 from the surface on the proximal end side of the illumination light transmission member 223. Moreover, the distal end of the illumination light transmission member 223 faces the window 221 of the case 220. In other words, when the case 220 is inserted in the oral cavity for use of the laryngeal camera, the distal end of the illumination light transmission member 223 reaches behind the uvula.

Without limiting, in the first embodiment, at the distal end of the illumination light transmission member 223, as illustrated in FIG. 4(B), a reflecting member 226 is provided. The reflecting member 226 is configured to reflect the illumination light to change an angle of the illumination light, to thereby direct the illumination light that has exited from the distal end of the illumination light transmission member 223 to the lower side of the inside of the body of the patient. The reflecting member 226 is a mirror or a prism, for example. When the distal end of the illumination light transmission member 223 can be directed to the direction of the larynx, the reflecting member 226 is not required, but when it is difficult to direct the distal end to the direction of the larynx, the reflecting member 226 is used to radiate the illumination light that has exited from a surface at the distal end of the illumination light transmission member 223 in the direction of the larynx through the reflecting member 226.

Meanwhile, without using the reflecting member 226, the light that exits from the illumination light transmission member 223 can be directed to the direction of the larynx. When the illumination light transmission member 223 is optical fibers, there may be adopted a method involving cutting the distal end of the illumination light transmission member 223 at an acute angle as illustrated in FIG. 4(C). The illumination light is reflected (for example, totally reflected) on the cut surface such that the illumination light that has exited from the illumination light transmission member 223 can be directed in a direction different from an extended line in a long direction of the illumination light transmission member 223, and more specifically, in the direction toward the larynx.

A description is given of usage and operation of the laryngeal camera unit 200 described above.

The laryngeal camera unit 200 forms the laryngeal camera in combination with the portable device 100. First, the laryngeal camera unit 200 and the portable device 100 are combined. The portable device 100 is inserted in the recess 213 of the main body portion 210 of the laryngeal camera unit 200. The insertion is performed such that a front surface of the portable device 100 abuts against the back surface of the plate portion 211. As a result, the display 102 of the portable device 100 is exposed from the recess 213 of the main body portion 210 of the laryngeal camera unit 200. Moreover, at this time, the lens 104 and the light 105 of the portable device 100 respectively enter states of being positioned right behind the first aperture 214 and the second aperture 215, which are formed in the plate portion 211 of the main body portion 210 of the laryngeal camera unit 200.

At this time, the portable device 100 is locked at edges on the back surface by the locking claws 212A and does not fall off the laryngeal camera unit 200.

This completes the laryngeal camera unit 200.

A user actuates the function of the camera of the portable device, which forms a part of the laryngeal camera unit 200, under a state of taking a moving image, and turns the light 105 into an ON state. The moving image taken by the camera is displayed on the display 102 of the portable device 100.

Under this state, the user inserts the case 220 of the laryngeal camera unit 200, which forms a part of the laryngeal camera, in the oral cavity of the patient, and inserts a tube included in the phlegm suction device from inside the oral cavity or a nasal cavity of the patient.

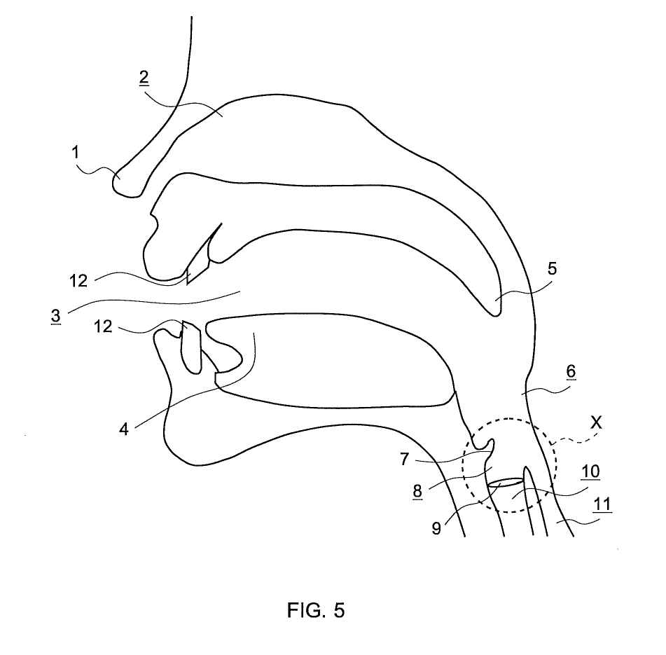

FIG. 5 is a cross-sectional view of a part of head and neck of a person. In FIG. 5, reference symbol 1 denotes an external nose, reference symbol 2 denotes a nasal cavity, and reference symbol 3 denotes an oral cavity. Reference symbol 4 denotes a tongue, reference symbol 5 denotes a uvula, reference symbol 6 denotes a pharynx (oropharynx), reference symbol 7 denotes epiglottis, reference symbol 8 denotes a larynx, reference symbol 9 denotes a glottis, reference symbol 10 denotes a trachea, reference symbol 11 denotes an esophagus, and reference symbol 12 denotes teeth. The region encircled by the broken line and indicated by reference symbol X is the place where phlegm tends to collect, and is generally a portion to be a phlegm suction position. Such phlegm suction position X is the region of the larynx 8.

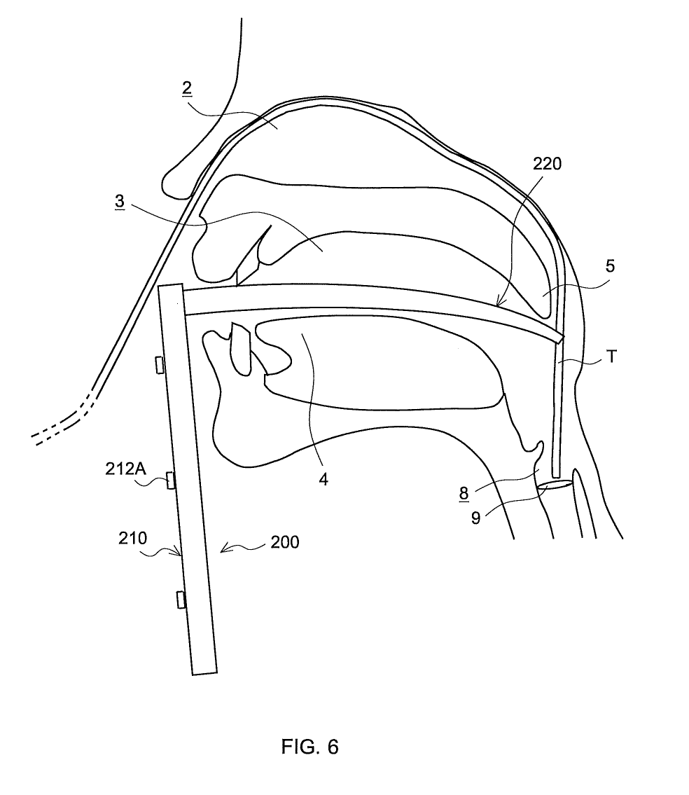

In this example, a tube T is inserted from the nasal cavity 2 until a distal end of the tube T reaches the phlegm suction position X (FIG. 6).

The distal end of the tube T and the phlegm suction position X are imaged by the laryngeal camera. Then the distal end of the case 220 of the laryngeal camera unit 200 reaches behind the uvula 5, and more specifically, the window 221 of the case 220 reaches behind the uvula 5. With the case 220 of the laryngeal camera unit 200 being bent, the distal end of the case 220 can reach behind the uvula 5 while avoiding interference of the case 220 with the tongue of the patient.

The illumination light required for the imaging is irradiated from the light 105 of the portable device 100. The light from the light 105 passes through the second aperture 215, which is formed in the plate portion 211 of the main body portion 210 of the laryngeal camera unit 200, to enter the illumination light transmission member 223 from the proximal end of the illumination light transmission member 223. The illumination light passes through the illumination light transmission member 223 to exit from the distal end side of the illumination light transmission member 223. In the first embodiment, the reflecting member 226 is present on the distal end side of the illumination light transmission member 223, and hence the illumination light that has exited from the distal end of the illumination light transmission member 223 is reflected by the reflecting member 226, and travels to the lower side of the body of the patient, that is, in the direction of the phlegm suction position X including the direction of the larynx 8. As a result, illumination of the phlegm suction position X is achieved.

The illumination light is reflected by the phlegm suction position X and the tube T to change into the image light. An image of the image light is taken by the camera of the portable device 100. In the first embodiment, the objective lens 224 and the reflecting member 225 are present at the distal end of the image light transmission member 222, and hence the image light is first reflected by the reflecting member 225, and travels to the objective lens 224. The image light having its diameter reduced by being refracted by the objective lens 224 enters the image light transmission member 222 from the distal end of the image light transmission member 222. The image light passes through the image light transmission member 222 and exits from the proximal end of the image light transmission member 222. Then, the image light passes through the first aperture 214, which is formed in the plate portion 211 of the main body portion 210 of the laryngeal camera unit 200, and enters the lens 104 of the portable device 100 such that an image thereof is taken by the image sensor (not shown) included in the portable device 100.

The image of the moving image that has been taken is displayed on the display 102 of the portable device 100. The user may check the position at which the phlegm is present and the position of the distal end of the tube T, or a relative positional relationship between the positions while adjusting a position and an angle of the portable device 100 with one hand. Meanwhile, the user may adjust the position of the distal end of the tube T by moving back and forth and rotating the tube T with another hand. When the distal end of the tube T has reached a position at which the phlegm can be sucked, the user may drive a suction pump of the phlegm suction device. The phlegm suction can be performed quickly and reliably when those actions are performed while checking with the display 102.

In the first embodiment, the portable device 100 includes the display 102, and the image taken by the image sensor of the portable device 100 is displayed on the display 102 substantially in real time, with the result that the user may perform the phlegm suction while checking the image. When the portable device 100 does not include the display, a display device including a display may be prepared separately from the portable device 100 as described above, and the image taken by the image sensor from the portable device 100 can be displayed on the display of the display device substantially in real time. Also in this manner, the user may perform the operation of the phlegm suction while checking the position at which the phlegm is present and the position of the distal end of the tube T, or the relative positional relationship between the positions during the phlegm suction.

When the phlegm suction is finished, the user may detach the portable device 100 from the laryngeal camera unit 200. Then, the portable device 100 may be used in originally intended usage.

### Second Embodiment

A laryngeal camera unit 200 according to the second embodiment is described.

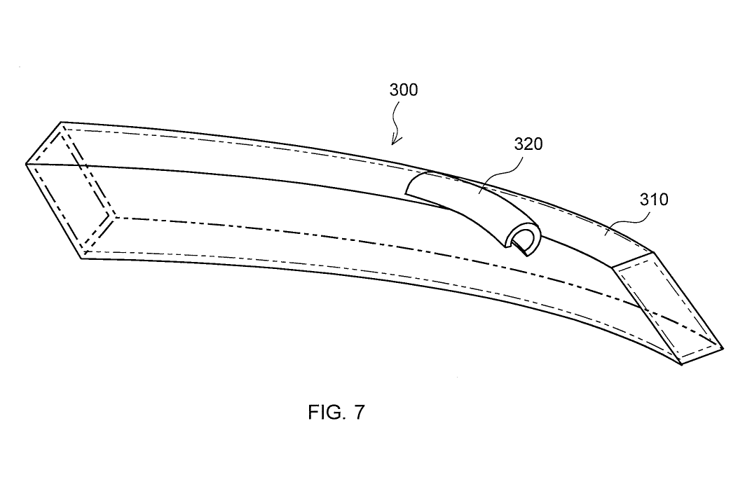

The laryngeal camera unit 200 according to the second embodiment is exactly the same as the laryngeal camera unit 200 according to the first embodiment, but further includes such a cover 300 as illustrated in FIG. 7. The cover 300 includes a cover main body 310, which is made of a thin material, and a guide member 320, which is provided to the cover main body 310. In the second embodiment, the cover main body 310 and the guide member 320 are integrally formed of a transparent resin having some stretchability.

The cover main body 310 is formed to be thin with a uniform thickness as a whole, for example, and has an inner shape that corresponds to an outer shape of the case 220 of the laryngeal camera unit 200. A rear end of the cover main body 310 is open, but the cover main body 310 is formed to be watertight in other parts. When the case 220 is inserted into the cover main body 310, the entire case 220 may be covered by the cover 300. However, it is sufficient when the cover main body 310 has a length that is enough to cover a portion of the case 220 to be inserted in the oral cavity with the cover main body 310, and it is not always required for the cover main body 310 to cover the total length of the case 220. In the second embodiment, the total length of the case 220 is covered by the cover main body 310.

When the case 220 is covered by the cover main body 310, the window 221 formed in the case 220 is also covered by the cover main body 310. In other words, when the laryngeal camera obtained by combining the laryngeal camera unit 200 according to the second embodiment and the portable device 100 is used, the image light and the illumination light are transmitted through the cover main body 310. In order to allow the transmission, it is required for at least a part of the cover main body 310 that covers the window 221 to be transparent. This can be easily achieved when the entire cover 300 is formed of a transparent resin. Moreover, particularly for the image light, when the image light is diffracted differently depending on the part of the cover 300 or the like when passing through the cover 300, distortion may occur in an image obtained by the image sensor of the portable device 100, for example. In order to prevent such a problem, at least the part of the cover main body 310 that covers the window 221 is required to have a uniform thickness. This can be achieved easily when the entire cover main body 310 has a uniform thickness.

The guide member 320 is configured to detachably fix the tube T. In the second embodiment, the guide member 320 is formed into a cross-sectional substantially C-shaped tube so that the tube T can be pushed into the guide member 320 while being deformed to fix the tube T, and the tube T can be pulled out from inside the guide member 320 while being deformed to cancel the fixation of the tube T.

The guide member 320 is configured to be bent, when the cover 300 is attached to the case 220 of the laryngeal camera unit 200, in a direction that is the same as the direction in which the case 220 is bent. It should be noted, however, that without limiting, in the second embodiment, a curvature of a curve of the guide member 320 is larger than a curvature of a curve of the case 220.

A description is given of usage and operation of the laryngeal camera unit 200 described above.

The laryngeal camera unit 200 according to the second embodiment also forms the laryngeal camera in combination with the portable device 100. The method of combination is similar to that in the case of the first embodiment.

It should be noted, however, that whatever the order with respect to the fixation between the portable device 100 and the laryngeal camera unit 200, in the second embodiment, the cover 300 is fixed to the case 220 of the laryngeal camera unit 200, and the tube T of the phlegm suction device is fixed to the guide member 320 of the cover 300. The tube T is fixed to the guide member 320 such that a length of the tube T beyond the guide member 320 is enough for the distal end of the tube T to reach the phlegm suction position X.

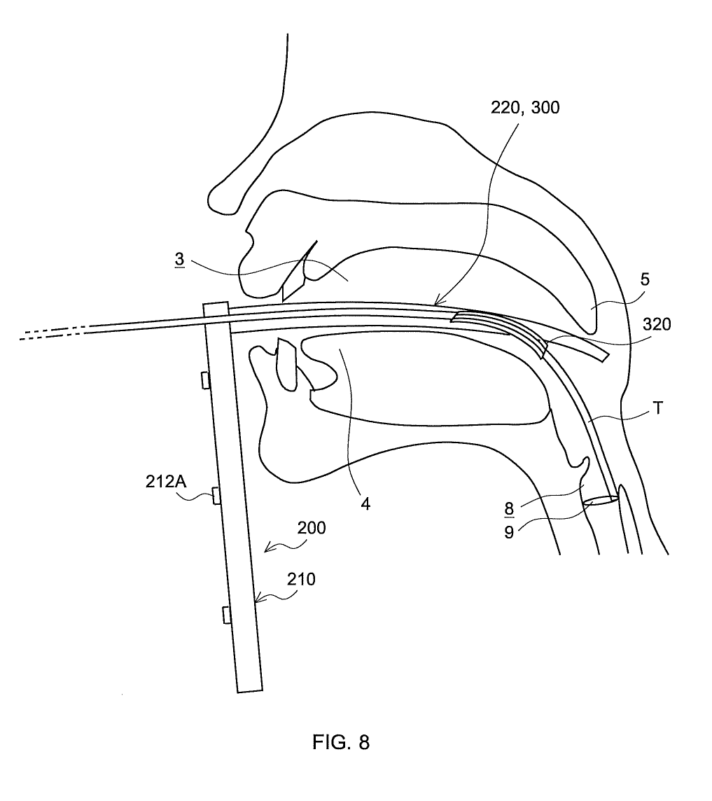

When a laryngeal camera including the laryngeal camera unit 200 according to the second embodiment is used, the tube T is inserted to the body of the patient together with the case 220 of the laryngeal camera from the oral cavity, where a smaller load is placed on the patient (FIG. 8).

The user operates the tube T together with the laryngeal camera. With the curvature of the guide member 320 being larger than the curvature of the case 220, the distal end of the tube T is headed further downward in the body of the patient, and hence the distal end of the tube T easily reaches the phlegm suction position X near the larynx 8, for example.

The second embodiment is the same as the first embodiment in that the phlegm suction position X of the patient can be illuminated with the illumination light from the light 105 of the portable device 100. The second embodiment is also the same as the first embodiment in that the phlegm suction position X and the distal end of the tube T can be imaged by the camera of the portable device 100.

The user may perform the phlegm suction of the patient while checking the image displayed on the display 102.

When the phlegm suction is finished, the user may detach the portable device 100 from the laryngeal camera unit 200, and also detach the cover 300 from the case of the laryngeal camera unit 200, and the tube T from the guide member 320 of the cover 300. The cover 300 may be not that expensive, and hence may be made disposable.

Also in the case of the second embodiment, when the phlegm suction is not performed, the portable device 100 may be used in the originally intended usage.

In the second embodiment, the guide member 320 is provided to the cover 300. Instead, the guide member 320 may be directly provided to the case 220. In this case, the guide member 320 may be formed integrally with the case 220.

Usage and operation of such a laryngeal camera unit 200 are not different from the usage and operation of the laryngeal camera unit 200 according to the second embodiment except that the case 220 is not covered by the cover 300.

### Third Embodiment

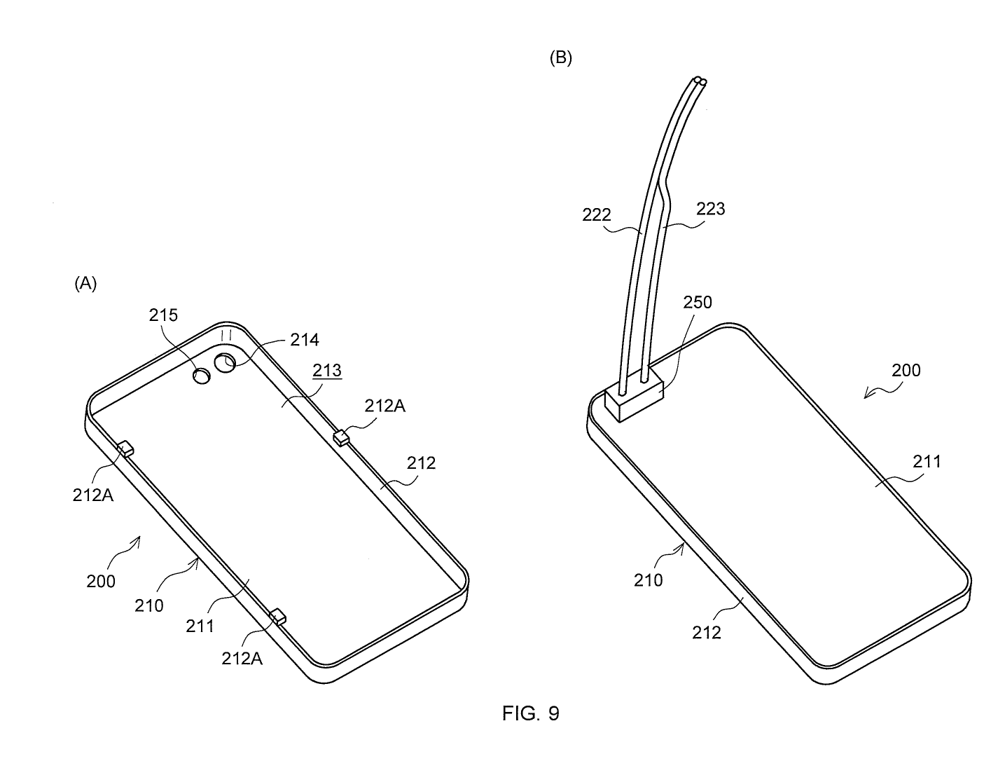

A laryngeal camera unit 200 according to the third embodiment is described with reference to FIG. 9.

The laryngeal camera unit 200 according to the third embodiment is substantially the same as the laryngeal camera unit 200 according to the first embodiment. The laryngeal camera unit 200 according to the third embodiment is different from the laryngeal camera unit 200 according to the first embodiment in that there is no case 220 configured to cover the image light transmission member 222 and the illumination light transmission member 223.

The laryngeal camera unit 200 according to the third embodiment is in a state in which the image light transmission member 222 and the illumination light transmission member 223 are, so to speak, bare. The image light transmission member 222 and the illumination light transmission member 223 are supported under a state of piercing through a support block 250, which is fixed to the plate portion 211 of the main body portion 210. A positional relationship on the proximal end side of the image light transmission member 222 and the illumination light transmission member 223 with respect to the plate portion 211 is the same as that in the first embodiment, and the third embodiment is also the same as the first embodiment in that the plate portion 211 has the first aperture 214 and the second aperture 215.

The image light transmission member 222 and the illumination light transmission member 223 in the third embodiment are similar to those in the first embodiment in that the image light transmission member 222 and the illumination light transmission member 223 are bent in the same manner as the case 220, which has been present in the first embodiment, in side view.

However, the illumination light transmission member 223 in the third embodiment extends along the image light transmission member 222 in a predetermined range on the distal end side of the illumination light transmission member 223, and a portion of the illumination light transmission member 223 is slightly bent at some midpoint in plan view. The degree in which the illumination light transmission member 223 is bent is in a range in which, when the illumination light transmission member 223 is the optical fibers, light transmission performance of the optical fibers is not affected, for example. Further, the portion on the distal end side in which the image light transmission member 222 and the illumination light transmission member 223 extend along each other are fixed to each other by an appropriate method, for example, by bonding.

The laryngeal camera unit 200 according to the third embodiment can be made thinner in the portion (bundle of the image light transmission member 222 and the illumination light transmission member 223) to be inserted in the oral cavity of the patient for the absence of the case, and hence is suitable for reducing the load on the patient. It should be noted, however, that it is conceivable that the image light transmission member 222 and the illumination light transmission member 223 lack in stiffness. In that case, it should be considered to increase the stiffness of the image light transmission member 222 and the illumination light transmission member 223 with a thin member that has high stiffness and extends along at least one of the image light transmission member 222 or the illumination light transmission member 223.

Usage and operation of such a laryngeal camera unit 200 are similar to those in the case of the first embodiment.

The guide member 320 described in the second embodiment may be attached to at least one of the image light transmission member 222 or the illumination light transmission member 223 of the laryngeal camera unit 200 according to the third embodiment.

1. A laryngeal camera unit, which is to be combined with a portable device to form a laryngeal camera,
the portable device being configured to be held by hand, and including an image sensor and a lens, which are configured to take an image of image light from a subject, and a light configured to emit illumination light for illuminating the subject,
the laryngeal camera being configured to take an image of a larynx,
the laryngeal camera unit comprising:
rod-shaped image light transmission means for receiving, from a distal end of the image light transmission means, the image light from the larynx, transmitting the image light along a long direction of the image light transmission means, and irradiating the lens of the portable device with the image light from a proximal end of the image light transmission means to allow the image sensor to take the image, the distal end being configured to reach behind a uvula when the larynx is imaged;
rod-shaped illumination light transmission means for receiving, from a proximal end of the illumination light transmission means, the illumination light from the light of the portable device, transmitting the illumination light along a long direction of the illumination light transmission means, and irradiating the larynx with the illumination light from a distal end of the illumination light transmission means, the distal end being configured to reach behind the uvula when the larynx is imaged, the illumination light transmission means being configured to extend along the image light transmission means at least in a predetermined range on the distal end side of the illumination light transmission means; and
a main body portion, to which the proximal end of the image light transmission means and the proximal end of the illumination light transmission means are attached, which includes fixing means for detachably fixing the main body portion to the portable device under a state of being positioned in a predetermined relative positional relationship, and which is to be fixed to the portable device so that the proximal end of the image light transmission means is positioned in front of and in proximity to the lens of the portable device, and so that the proximal end of the illumination light transmission means is positioned in front of and in proximity to the light of the portable device.

2. A laryngeal camera unit according to claim 1, wherein the image light transmission means and the illumination light transmission means are bent to correspond to a curve of a palate in a vertical direction of the palate.

3. A laryngeal camera unit according to claim 1, wherein the image light transmission means and the illumination light transmission means are fixed to each other.

4. A laryngeal camera unit according to claim 1, wherein the image light transmission means and the illumination light transmission means are housed in a case, which is configured to house the image light transmission means and the illumination light transmission means under a state of allowing light to pass through the distal end and the proximal end of each of the image light transmission means and the illumination light transmission means.

5. A laryngeal camera unit according to claim 4, wherein the case is bent to correspond to a curve of a palate in a vertical direction of the palate.

6. A laryngeal camera unit according to claim 1, wherein at least one of the image light transmission means or the illumination light transmission means includes a tube fixing member configured to detachably fix a flexible tube, which is included in a phlegm suction device, under a state in which a distal end of the flexible tube is positioned in an imaging range of the image sensor.

7. A laryngeal camera unit according to claim 2, wherein at least one of the image light transmission means or the illumination light transmission means includes a tube fixing member capable of detachably fixing a flexible tube, which is included in a phlegm suction device, and
wherein the tube fixing member is configured to fix the flexible tube with the flexible tube in a portion fixed by the tube fixing member being bent at a curvature that is larger than curvatures imparted to the image light transmission means and the illumination light transmission means so that a distal end of the flexible tube is headed to the larynx.

8. A laryngeal camera unit according to claim 4, wherein the case includes a tube fixing member capable of detachably fixing a flexible tube, which is included in a phlegm suction device, under a state in which a distal end of the flexible tube is positioned in an imaging range of the image sensor.

9. A laryngeal camera unit according to claim 5, wherein the case includes a tube fixing member configured to detachably fix a flexible tube, which is included in a phlegm suction device, and
wherein the tube fixing member is configured to fix the flexible tube with the flexible tube in a portion fixed by the tube fixing member being bent at a curvature that is larger than a curvature imparted to the case so that a distal end of the flexible tube is headed to the larynx.

10. A laryngeal camera unit according to claim 4, further comprising a cover, which is configured to cover at least a portion of the case to be inserted in an oral cavity, which is detachably attachable to the case, and in which at least portions for allowing passage of the image light and the illumination light are transparent.

11. A laryngeal camera unit according to claim 10, wherein the cover includes a tube fixing member configured to detachably fix a flexible tube, which is included in a phlegm suction device, under a state in which a distal end of the flexible tube is positioned in an imaging range of the image sensor.

12. A laryngeal camera unit according to claim 1, wherein the portable device includes a smartphone or a tablet computer.

13. A laryngeal camera unit according to claim 1, wherein the laryngeal camera includes a phlegm suction camera, which is to be used to assist in phlegm suction by imaging a phlegm suction position.

14. A laryngeal camera set, comprising in combination:
the laryngeal camera unit of claim 1; and
the portable device.

15. A cover, which is included in the laryngeal camera unit of claim 10.
